(19) United States
(12) Patent Application Publication
(10) Pub. No.: US 2020/0375531 A1
(43) Pub. Date: Dec. 3, 2020

(54) THERMOACOUSTIC IMAGING METHODS AND SYSTEMS UTILIZING PARALLEL PHASED ARRAY TRANSMISSION ELEMENTS

(71) Applicant: ENDRA Life Sciences Inc., Ann Arbor, MI (US)

(72) Inventors: Christopher Nelson Davis, Ann Arbor, MI (US); Paul A. Picot, London (CA)

(73) Assignee: ENDRA Life Sciences Inc., Ann Arbor, MI (US)

(21) Appl. No.: 16/423,847

(22) Filed: May 28, 2019

(51) Int. Cl. A61B 5/00; A61B 5/05; G01N 29/24

(52) U.S. Cl. CPC A61B 5/4872; G01N 29/2431; A61B 5/05; A61B 5/0093

(57) ABSTRACT

A method for providing an image of a subject by utilizing an array of transmission elements in a thermoacoustic imaging apparatus. The method includes providing an ultrasound image of the subject, determining a body anatomy from the ultrasound image, matching the body anatomy with at least one body model of a plurality of body models, utilizing shaped illumination fields from the at least one body model to adjust the parameters of a plurality of radio-frequency applicator elements upon the subject that will optimize the energy delivery and uniformity of illumination of a thermoacoustic stimulus in a region of interest (ROI), while also minimizing the thermoacoustic stimulus in other regions which could generate unwanted thermoacoustic artifacts that could interfere with the thermoacoustic signal from the ROI, wherein the radio-frequency applicator elements operate with independent, adjustable parameters, and utilizing the radio-frequency applicator elements to perform a thermoacoustic measurement of the subject.

### FIELD

The present disclosure describes methods and systems of utilizing parallel phased array transmission elements for thermoacoustic imaging.

### BACKGROUND

In traditional ultrasound medical imaging, or sonography, a single array of ultrasound transducers (herein sometimes called a transmit-receive array) operates both to transmit and receive ultrasound energy. Ultrasound transducer elements transmit ultrasound waves into an object (e.g., tissue). The transmitted energy is scattered and reflected by the tissue, and the scattered and reflected ultrasound energy is received by the same ultrasound transducer elements. The ultrasound transducer converts received ultrasound energy to electrical signals. The received ultrasound signals are analyzed and interpreted through signal processing, generally providing information on location of structures within the tissue.

In medical ultrasound imaging, ultrasound pulses are used in a manner similar to radar, where a pulse is transmitted, and then echoes are received from reflections and from scatter within tissue. In radar (RAdio Detection And Ranging), a short pulse of an electromagnetic (radiofrequency or microwave) carrier wave is transmitted, and echoes or reflections are detected by a receiver, typically co-located with the transmitter. The range of radar is limited by the received signal energy. Analogously, in ultrasound medical imaging, strong, short electrical pulses transmitted by the ultrasound system drive the transducer at a desired frequency in order to achieve good range resolution. The two-way time of flight of received echoes yields range information, and the strength of the received echoes provides information on acoustical impedance (e.g., when a transmitted pulse encounters a structure within tissue with a different density, and reflects back to the transducer). With knowledge of the direction of the transmitted pulse, an ultrasound image, or sonogram, is created. In ultrasound medical imaging, the maximum transmitted power is limited by the voltage tolerated in the system electronic components and by the peak intensity permitted by safety considerations pertaining to tissue exposure. As in radar, the range is limited by the received signal versus background noise, which is in turn limited by total pulse energy.

Thermoacoustic imaging, sometimes called photoacoustic or optoacoustic imaging, is a technology used in characterizing and imaging materials based on their electromagnetic and thermal properties, having applications in nondestructive testing, clinical diagnostics, medical imaging, life sciences, and microscopy. Thermoacoustic imaging uses short pulses of electromagnetic (EM) energy, i.e., the excitation energy, to rapidly heat features within an object that absorb the EM energy (excitation sites). The EM energy is typically in the radio-frequency (RF) range. This rapid heating causes the material (e.g., tissue) to increase in pressure slightly, inducing acoustic pulses that radiate from the excitation site as an ultrasound wave. These acoustic pulses are detected using acoustic receivers, such as an array of ultrasound transducers, located at the material's surface. The resulting measurements are analyzed and interpreted through signal processing using time-of-flight and related algorithms, which reconstruct the distribution of absorbed EM energy, sometimes called thermoacoustic computed tomography (TCAT). The result can be displayed to the user as depth profile plots, or as 2-, 3-, or 4-dimensional images.

There are different requirements for clinical ultrasound transducers operating in transmit-receive mode versus receive-only ultrasound transducers employed in thermoacoustic imaging. Clinical ultrasound transducer arrays are constructed and optimized to operate in both transmit and receive ultrasound modes. These ultrasound transducers require high operating efficiency in transmitting and receiving ultrasound energy, which is not a requirement of receive-only transmitters used in thermoacoustic imaging. Clinical ultrasound transducers typically use a lens to provide an optimal depth of focus and are designed with an optimized frequency of operation. Traditional ultrasound imaging relies upon narrow band reception for image resolution.

By contrast, in thermoacoustic imaging, it is important for the receive-only transducers to receive and process a wide band of frequencies. Thermoacoustic transducer elements and arrays are designed to operate with a high sensitivity in receive-only mode, whereas receive-only transducers do not have to meet the transmission efficiency requirements of transmit-receive elements and arrays. Thermoacoustic image resolution is determined by frequency of the acoustic signal. This frequency is determined by characteristics of the material being imaged, not by the frequency of the emitted electromagnetic energy (or excitation energy). To be able to discriminate a range of materials properties in thermoacoustic imaging (e.g., small and large size structures, imaging shallow materials and deep materials), wide reception bandwidth is critical. A reception bandwidth on the order of 3-6 MHz is considered a fairly wide range, and higher bandwidths are desirable.

One consideration in image formation in both ultrasound imaging and in thermoacoustic imaging is transducer geometry, e.g., geometry and configuration of transducer arrays. Different transducer geometries, such as single focused transducer, linear arrays, and two-dimensional arrays, are capable of different modes of image formation. Depending in part on the transducer geometry, the imaging system may image, for example, single lines, two-dimensional regions, or three-dimensional volumes. The imaging operation also may employ scanning, or motion of the transducers or transducer arrays, to adapt transducer operation to different modes of imaging.

Traditional clinical ultrasound technology indicates locations of features within a tissue or other material, but provides no functional characteristics. On the other hand, thermoacoustic imaging combines absorption contrasts achieved through interaction of the imaged material with the EM excitation energy, with fine ultrasound resolutions characteristic of acoustic reception, thereby enabling deep penetration in in vivo imaging. Thermoacoustic technology can detect dynamic features and can measure various functional characteristics of anatomy.

### SUMMARY

Embodiments disclosed herein utilize a radiofrequency electric field emitter and multiple parallel and independent transmit channels, each comprising a waveform generator, power amplifier, tune and match circuitry, and one or more transmitter array elements. Each channel can be independently adjustable in at least amplitude and phase, and more generally also adjustable in waveform, frequency, and polarization. Further, each applicator element can be adjustable to match a complex impedance of a body (e.g., patient) at its location, to maximize power transmission and minimize reflected power to optimize the image.

In one embodiment, a method for providing an image of a subject by utilizing an array of transmission elements in a thermoacoustic imaging apparatus comprises generating, by a processor, an ultrasound image of the subject; identifying, by the processor, a body anatomy from the ultrasound image; matching, by the processor, the body anatomy with at least one body model of a plurality of body models; adjusting, by the processor, parameters of a plurality of independently-adjustable radio-frequency applicator elements based upon at least one matching body model to optimize the energy delivery and uniformity of illumination of a thermoacoustic stimulus in a specific region of interest, and minimize thermoacoustic stimuli outside of the specific region of interest; and performing, by the processor, a thermoacoustic measurement of the subject utilizing the radio-frequency applicator elements. Adjusting parameters of a plurality of independently-adjustable radio-frequency applicator elements may comprise steering a beam from a first location to a second location based on the body anatomy. Adjusting parameters may comprise calculating a revised parameter based on a depth, width, and volume of the matching body model. Adjusting parameters may comprise identifying parameters in a lookup table based upon the matching body model. The parameters can be automatically adjusted for transmission upon matching with at least one body model. Adjusting parameters can use amplitude and phase values for each radio-frequency applicator element from a simulation of the matching body model. Each radio-frequency applicator element can be adjusted simultaneously. The parameters of each applicator element can be determined by a respective shaped illumination field. The radio-frequency applicator elements can be arranged in a circular array, a symmetrical array, or with an offset from a center location. The parameters can include amplitude, phase, frequency, polarization, waveform, and/or input impedance. The thermoacoustic measurement can be utilized to calculate a value within the subject, such as a fat concentration.

In another embodiment, a thermoacoustic imaging system comprises a set of applicator elements, each applicator element driven by an independent amplifier, wherein each independent amplifier has an adjustable phase and amplitude for each applicator element channel; and the independent amplifier of each applicator element configured to adjust phase and amplitude for each applicator element of each channel to optimize a uniformity of energy deposition over a target volume and to minimize energy disposition in other volumes to minimize thermoacoustic artifacts, and configured to adjust each applicator element to maximize energy absorption in the target volume, thereby steering the set of applicator elements to the target volume. The thermoacoustic imaging system can include an ultrasound transducer configured to generate an ultrasound image of the target volume, wherein the independent amplifier adjusts each applicator element based upon the ultrasound image of the target volume.

In yet another embodiment, a method for providing an image of a subject by utilizing an array of transmission elements in a thermoacoustic imaging apparatus comprises: providing an ultrasound image of the subject; determining a body anatomy from the ultrasound image; matching the body anatomy with at least one body model of a plurality of body models; utilizing shaped illumination fields from the at least one body model to adjust the parameters of a plurality of radio-frequency applicator elements upon the subject that will optimize the energy delivery and uniformity of illumination of a thermoacoustic stimulus, wherein the radio-frequency applicator elements operate with independent, adjustable parameters, further wherein the parameters include at least an amplitude, phase, frequency, polarization, waveform, or input impedance, and further wherein the parameters of each applicator element are determined by a respective shaped illumination field; and utilizing the radio-frequency applicator elements to perform a thermoacoustic measurement of the subject.

In a separate embodiment, the parameters of each applicator element are determined by a respective shaped illumination field.

In a separate embodiment, the parameters of amplitude and phase are adjusted to focus the field amplitude at a particular region of interest for thermoacoustic measurements while minimizing the field amplitude at the interface between the applicator and the subject's skin. which significantly reduces the thermoacoustic signal generated at the surface of the applicator that causes unwanted artifacts in the thermoacoustic measurement from the region of interest.

In a separate embodiment, the phase and amplitude of the applicator elements are dynamically adjusted to scan the illumination field over a specified region of interest(s) on the subject.

In a separate embodiment, the frequency of the elements can be adjusted to remove undesirable hot spots or nulls resulting from the specific anatomy of the subject that could affect thermoacoustic image quality

In a separate embodiment, one of the parameters is a polarization.

In a separate embodiment, one of the parameters is a waveform.

In a separate embodiment, the input impedance of the element is adjusted to maximize the thermoacoustic response for a given subject's anatomy and region of interest.

In a separate embodiment, the thermoacoustic measurement is utilized to calculate a value within the subject.

In a separate embodiment, value is a fat concentration.

### BRIEF DESCRIPTION OF THE DRAWINGS

Embodiments will now be described more fully with reference to the accompanying drawings.

FIG. 1 is a schematic view of an imaging system, according to an embodiment.

FIG. 2 is a schematic view of an imaging system with a plurality of radio-frequency applicator elements, according to an embodiment.

FIG. 3 is an embodiment of the method discussed in this disclosure, according to an embodiment.

FIGS. 4A to 4C are schematic views of an imaging system with a plurality of radio-frequency applicator elements, according to an embodiment.

FIG. 5 shows thermoacoustic artifact generation at a transducer-skin boundary with a fat/muscle signal of interest, according to an embodiment.

FIG. 6 shows thermoacoustic artifact generation at the applicator-skin boundary and a muscle/liver signal of interest, according to an embodiment.

### DETAILED DESCRIPTION

As in optical imaging, thermoacoustic image contrast depends on differences in energy absorption in a target object, due to intrinsic tissue properties or an extrinsic contrast agent. The amount of energy absorbed and signal produced depends on both the target object material characteristics and the amplitude of the illuminating radiofrequency field. A uniform illumination field is preferred so that differences in object contrast dominate the received signal or reconstructed image. Non-uniform illumination causes interpretation errors and hampers quantitative analysis. It also imposes higher dynamic range and linearity requirements on the detector system.

In radio-frequency thermoacoustics, frequencies lower than a few hundred megahertz (MHz) are poorly absorbed. Higher radio frequencies in the ultra high frequency (UHF) range (300-1000 MHz) are preferred, because of their higher absorption and greater signal production in tissue. Microwave frequencies, those much higher than 1000 MHz, are strongly absorbed and do not penetrate to useful depths in tissue.

Higher radio frequencies have shorter wavelengths, which are further shortened by the high dielectric constant of water in tissue. The useful UHF radio frequencies have wavelengths in tissue approximately in the range of human body part dimensions.

When an absorbing body with a high dielectric constant is illuminated by energy with a wavelength of approximately its dimensions, local non-uniformities in the illuminating field will occur in the body due to absorption and so-called “cavity” effects caused by resonance or “echoing” within the body. This non-uniformity is, in general, dependent on the body geometry and material characteristics.

The non-uniformity of illumination can be corrected by using a shaped or tailored illumination field. Illumination is adjusted in amplitude and phase over the surface of the body such that the field inside the body is made to be uniform throughout the imaging region of interest. This can be accomplished by a plurality of similar applicator elements distributed over or near the surface of the body and operating in parallel, but with independent amplitude and phase. The goodness of correction, finesse, or uniformity of the resulting field depends on the number and distribution of the applicator elements and the degree of control of phase and amplitude exercised on each element.

The use of multiple parallel transmitter channels solves the additional problem of high power handling requirements. In the case of a one- or two-transmitter system, the transmitter is called upon to provide high power, typically 10 kilowatts or more. Devices capable of producing this high power are bulky, rare, prone to failure, and expensive. Alternatively, multiple smaller devices can be combined with an additional power combiner component, but the final high power output must still be accommodated in the transmit element, connectors, and cabling. The parallel transmitter approach described herein uses multiple identical independent low power transmitters but no power combiner component. This solves the problem of high power handling by using multiple low-power components instead.

In x-ray computed tomography imaging, an analogous operation to reduce the dynamic range requirements of the detector is performed by a “bow-tie filter” of aluminum or graphite placed between the x-ray source and the patient. In previous versions, a less-practical water bath was used for this purpose.

In ultra high field magnetic resonance imaging (MRI), which uses similar radio frequencies but much higher pulse energies, an analogous problem of non-uniformity exists, causing both image non-uniformity and ‘hot spots’ of heating in tissue. The specific absorption rate (SAR) heating non-uniformity problem is an area of active research in the MRI community. Image non-uniformity is addressed by several approaches, including specific coil design and (partially) other acquisition methods such as Sensitivity Encoding (SENSE) and SiMultaneous Acquisition of Spatial Harmonics (SMASH). Multiple transmitter channels, generally called “RF shimming,” is a commercially-available feature on some MRI systems (e.g., Philips® MultiTransmit, which uses just two channels and has been shown to be sufficient for that application).

In optical imaging, an analogous operation uses adaptive optics for wavefront correction to de-blur images. Originally developed using deformable mirrors to sharpen astronomical images, optical components for this purpose are now commercially available.

In ultrasound imaging, image blurring is also caused by wavefront aberrations, due to differing speeds of sound in tissue. Here, wavefront corrections in amplitude and phase can be made in both the transmit and receive, with some additional hardware components. However, in practice, corrections are typically made only on the received signal.

In phased-array and synthetic aperture radar imaging, similar phase and amplitude adjustments over a plurality of elements are used as part of the image formation process but do not appear to use the phase and amplitude adjustments to correct the transmitted beam for absorption effects in the target object. In radar, the object does not exhibit cavity or resonance effects, so corrections are not made for these effects.

The present disclosure discusses systems and methods that utilize a radiofrequency electric field emitter and uses multiple parallel and independent transmit channels, each comprising a waveform generator, power amplifier, tune and match circuitry, and one or more transmitter array elements. Each channel is independently adjustable in at least amplitude and phase, and more generally also adjustable in waveform, frequency, and polarization. Further, each applicator element can be adjustable to match a complex impedance of a body (e.g., patient) at its location, to maximize power transmission and minimize reflected power to optimize the image.

By adjusting the parameters of each of the multiple parallel and independent channels, the systems and methods can optimize the energy delivery and uniformity of illumination of a thermoacoustic stimulus in thermoacoustic imaging. The systems and methods employing this design will have applications in other fields that require tailoring of RF fields, including the more general case of specifically-shaped field intensities for use in selective heating or targeting of body tissue or other material.

In an effort to obtain a more uniform beam from an object that may cause reflects, hot spots, or null zones, the systems and methods use the following elements: the use of multiple RF applicator elements or sets of elements, each driven by an independent amplifier; an independently-adjustable phase of each amplifier and element channel; an independently-adjustable amplitude of each amplifier and element channel; an adjustment of the phase and amplitude of each channel to optimize the uniformity of energy deposition over a defined target volume; and an independent adjustment of tune and match of each applicator element or element set to maximize energy absorption in the target volume.

Although the embodiments described herein relate to thermoacoustic imaging, embodiments of the systems and methods can also be used for tailoring an electric field within a volume that can be used to target specific sub-volumes for selective heating, such as hyperthermia therapy or drug-release applications. The systems and methods can also be used for a field analysis approach or algorithm and can be useful for controlling local heat deposition (SAR) in magnetic resonance imaging applications.

In one embodiment, a configuration is freely-adjustable in phase and amplitude, and optionally also in waveform, frequency, and polarization. In one configuration, waveform, and frequency are common to all channels, and polarization is dictated by the physical element structure. Alternate configurations could alter any or all parameters or any combination of these parameters.

In one embodiment, a configuration defines the parameters electronically, and can be adjusted under control of a software algorithm. Alternate configurations may use predefined or fixed parameters, which may be defined by physical circuit elements or by programmable hardware or some combination of the two.

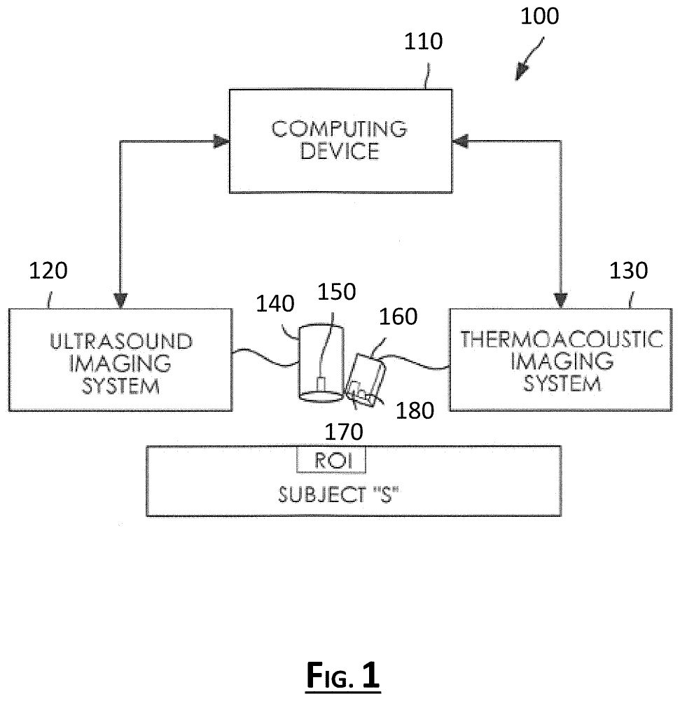

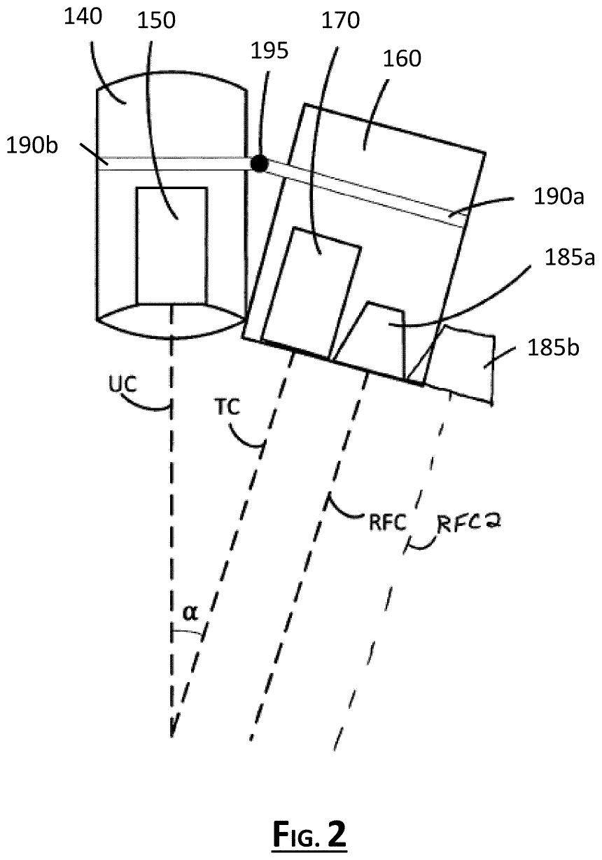

An artifact can occur at any type of interface where two dissimilar tissues or materials are in contact with one another. The dissimilar tissues or materials create a thermoacoustic bipolar signal at the interface. The thermoacoustic signal has a ring down time that depends on the characteristics of the transducer and the strength of the thermoacoustic signal. It is desirable to minimize the thermoacoustic artifact to limit signal interference with the thermoacoustic signal from the region of interest (ROI) that occurs when the thermoacoustic signal from the ROI arrives at the transducer while ring down of the thermoacoustic artifact is still occurring. For example (FIG. 1), a thermoacoustic signal can be generated at the interface between the transducer and the skin. This thermoacoustic signal can propagate into the transducer and overlap in time with the signal that would be generated at the fat and muscle interface, which can be 5 mm or less from the surface of the skin depending on patient anatomy. In another example (FIG. 2), a large thermoacoustic signal is typically generated between the surface of the transmitter element and the skin due the large electric field generated at the aperture of a single element transmitter and the conductivity mismatch between the transmitter and skin. This thermoacoustic signal can reflect off tissue boundaries inside the body, such as a fat-muscle boundary, and, depending on patient anatomy and relative angles between the transducer and transmitter, can overlap in time with signals from the ROI, such as the muscle-liver boundary.

Turning now to FIG. 1, an imaging system 100, according to an embodiment. The imaging system 100 comprises a programmed computing device 110 communicatively coupled to an ultrasound imaging system 120 and to a thermoacoustic imaging system 130. The ultrasound imaging system 120 and thermoacoustic imaging system 130 are configured to obtain ultrasound image data and thermoacoustic image data, respectively, of a tissue region of interest (ROI) associated with a subject S.

The programmed computing device 110 can be a personal computer or other suitable processing device comprising, for example, a processing unit comprising one or more processors, non-transitory system memory (volatile and/or non-volatile memory), other non-removable or removable memory (e.g., a hard disk drive, RAM, ROM, EEPROM, CD-ROM, DVD, flash memory, etc.) and a system bus coupling the various computer components to the processing unit. The computing device 110 may also comprise networking capabilities using Ethernet, Wi-Fi, and/or other suitable network format, to enable connection to shared or remote drives, one or more networked computers, or other networked devices. One or more input devices, such as a mouse and a keyboard (not shown), are coupled to the computing device 110 for receiving operator input. A display device (not shown), such as one or more computer screens or monitors, is coupled to the computing device 110 for displaying one or more generated images that are based on ultrasound image data received from the ultrasound imaging system 120 and/or the thermoacoustic image data received from thermoacoustic imaging system 130.

The ultrasound imaging system 120 comprises an acoustic receiver in the form of an ultrasound transducer 140 that houses one or more ultrasound transducer arrays 150 configured to emit sound waves into the ROI of the subject S. The sound waves directed into the ROI of the subject echo off tissue within the ROI, with different tissues reflecting varying degrees of sound. Echoes that are received by the one or more ultrasound transducer arrays 150 are processed by the ultrasound imaging system 120 before being communicated as ultrasound image data to the computing device 110 for further processing and for presentation as ultrasound images that can be interpreted by an operator. In this embodiment, the ultrasound imaging system 120 utilizes B-mode ultrasound imaging techniques assuming a nominal speed of sound of 1,540 m/s.

The thermoacoustic imaging system 130 comprises an acoustic receiver in the form of a thermoacoustic transducer 160. The thermoacoustic transducer 160 houses one or more thermoacoustic transducer arrays 170 as well as a radio-frequency (RF) applicator element 185a of thermoacoustic transducer arrays 180. It will be appreciated that the RF applicator 185a may be housed separately from the thermoacoustic transducer 160. The RF applicator 185a is configured to emit short pulses of RF energy that are directed into tissue within the ROI of the subject S. In this embodiment, the RF applicator 185a has a frequency between about 10 Mhz and 100 GHz and has a pulse duration between about 0.1 nanoseconds and 5000 nanoseconds, and more particularly between about 50 nanoseconds to 5000 nanoseconds. The RF energy pulses delivered to the tissue within the ROI heat the tissue thereby to induce acoustic pressure waves that are detected by the thermoacoustic transducer 160. The acoustic pressure waves that are detected by the thermoacoustic transducer 160 are processed and communicated as thermoacoustic image data to the computing device 110 for further processing and for presentation as thermoacoustic images that can be interpreted by the operator.

The imaging system can independently adjust parameters the RF applicator elements of the thermoacoustic transducer array to steer a beam based on a shaped illumination field for a body anatomy of a subject as viewed by an ultrasound image. The parameters can include amplitude, phase, frequency, polarization, waveform, and/or input impedance.

FIG. 2 is a schematic view of an imaging system with a plurality of RF applicator elements. In one embodiment, each RF applicator element 185a, 185b has a separate channel for transmitting an RF signal. Each RF applicator element and channel can be configured to have its own variable attenuator and phase adjuster. Each channel can have a frequency from an RF source adjusted. Each RF applicator element and channel can use tuning screws to change an input impedance.

Each channel or applicator element can have an amplifier, variable attenuator, phase shifter, and/or any other components for adjusting parameters of the transmission. Although this example has separate and independent channels for each applicator element, the imaging system can be configured such that one or more RF applicator elements rely or depend on another one or more RF applicator elements. For example, the dependency can be based upon a mathematical relationship such that only a pair of applicator elements would have a single amplitude adjustment. In another example, one applicator element can depend upon another applicator element such that one applicator element is always half of the amplitude of the other applicator element. Such a configuration could allow these channels to share hardware (e.g., variable attenuator or phase adjuster) or reduce the amount of hardware.

In this embodiment, the spatial relationship between the one or more ultrasound transducer arrays 150 and the one or more thermoacoustic transducer arrays 170 is such that the centerline of the one or more thermoacoustic transducer arrays 170 is set at a known angle α with respect to the centerline (also known as the axial axis or ultrasound transducer array beam axis) of the one or more ultrasound transducer arrays 150. The spatial relationship between the one or more thermoacoustic transducer arrays 180, first RF applicator 185a, and second RF applicator 185b is such that the centerlines of first RF applicator 185a and second RF applicator 185b are spaced-apart and generally parallel to the centerline of the one or more thermoacoustic transducer arrays 170.

The imaging system 100 utilizes the known spatial relationship between the one or more ultrasound transducer arrays 150 and the one or more thermoacoustic transducer arrays 170 to increase the precision and accuracy of thermoacoustic imaging. A coordinate system of the one or more ultrasound transducer arrays 150 of the ultrasound transducer 140 and a coordinate system of the one or more thermoacoustic transducer arrays 170 of the thermoacoustic transducer 170 are mapped by the computing device 110 so that acquired ultrasound and thermoacoustic images can be registered. Alternatively, the thermoacoustic imaging system 130 may use of the one or more ultrasound transducer arrays 150 of the ultrasound transducer 140 by disconnecting the one or more ultrasound transducer arrays 150 from the ultrasound transducer 140 and connecting the one or more ultrasound transducer arrays 150 to the thermoacoustic transducer 160. This coordinate mapping between the one or more ultrasound transducer arrays 140 and the one or more thermoacoustic transducer arrays 170 is optional.

As shown in FIG. 2, the thermoacoustic transducer 160 is mechanically connected to the ultrasound transducer 140 using a connector 195. In this embodiment, the connector 195 connects a band or strap and is made of a rigid material, such as metal, plastic, or the like. A band 190 extends around an outside surface of the thermoacoustic transducer 160 and connects via connector 195 to a band 190b around the ultrasound transducer 140. In this embodiment, the bands 190a, 190b are mechanically connected to the outside surface of the thermoacoustic 150, transducer 160, and the ultrasound transducer 140 using a fastener, such as a screw, clamp, or the like. As will be appreciated, in other embodiments, the bands 190a, 190b may be mechanically connected to the outside surface of the thermoacoustic transducer 160 and the ultrasound transducer 140 using adhesive, friction, etc.

The connector 195 and bands 190a, 190b are configured such that the spatial relationship between the one or more ultrasound transducer arrays 150, the one or more thermoacoustic transducer arrays 170, first RF applicator 185a, and second RF applicator 185b are known. In other words, the connector 810 is used to fix the spatial relationship between the one or more ultrasound transducer arrays 150, the one or more thermoacoustic transducer arrays 170, first RF applicator 185a, and second RF applicator 185b. In this embodiment, the spatial relationship is set using a centerline of the one or more ultrasound transducer arrays 150, the one or more thermoacoustic transducer arrays 170, first RF applicator 185a, and second RF applicator 185b. Each centerline is defined as being a mid-point of an area of the respective array(s) or face. The imaging system 100 utilizes the known spatial relationship between the one or more ultrasound transducer arrays 150 and the one or more thermoacoustic transducer arrays 170 to increase the precision and accuracy of thermoacoustic imaging.

In one embodiment, the system may include an ultrasound transmit-receive transducer array and a thermoacoustic receive-only transducer array, which can assume a wide variety of two-dimensional array geometries, such as linear, curved linear, circular, square, and rectangular array geometries. Individual elements of the ultrasound transmit-receive transducer array and the thermoacoustic receive-only transducer array can have various shapes, such as square, circular, elliptical, rectangular, and polygon. The transducer arrays can have a single applicator element, a few elements, dozens of elements, hundreds of elements, or thousands of elements.

The elements of the ultrasound transmit-receive array can be configured in various geometries with the elements of the thermoacoustic receive-only array. For example, elements of a receive-only array can surround elements of a transmit-receive array, such as a linear array of receive-only elements on each side of a linear transmit-receive array. In another example, elements of a linear receive-only array can align with elements of a linear transmit-receive array. In yet another example, elements of a receive-only array can be interspersed with elements of a transmit-receive array in a regular pattern (e.g., alternating) or irregular pattern (e.g., random or sparse array of one type interspersed with dense array of the other array).

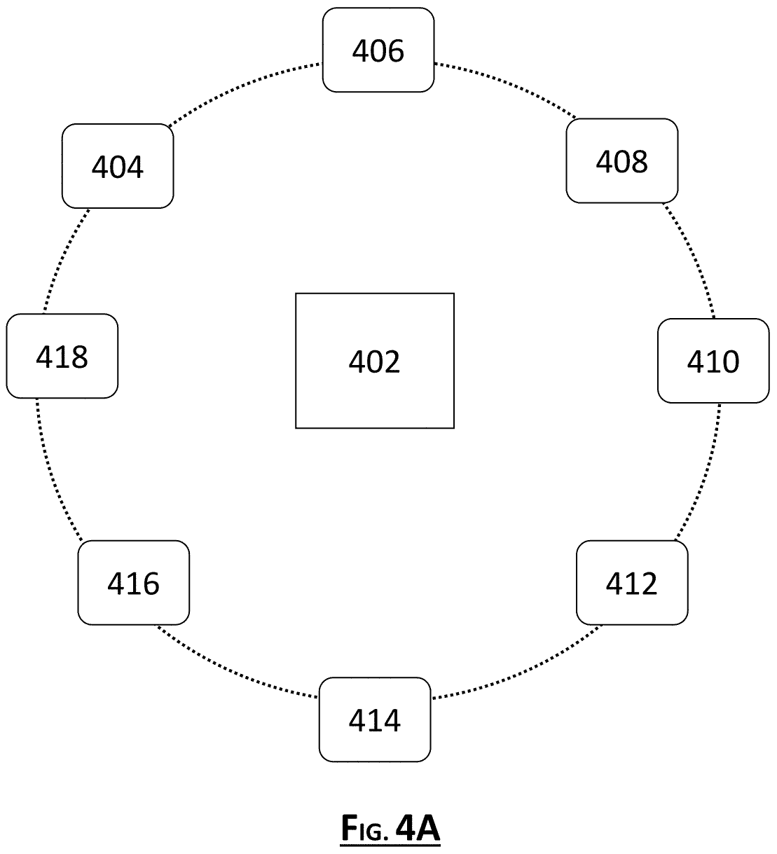

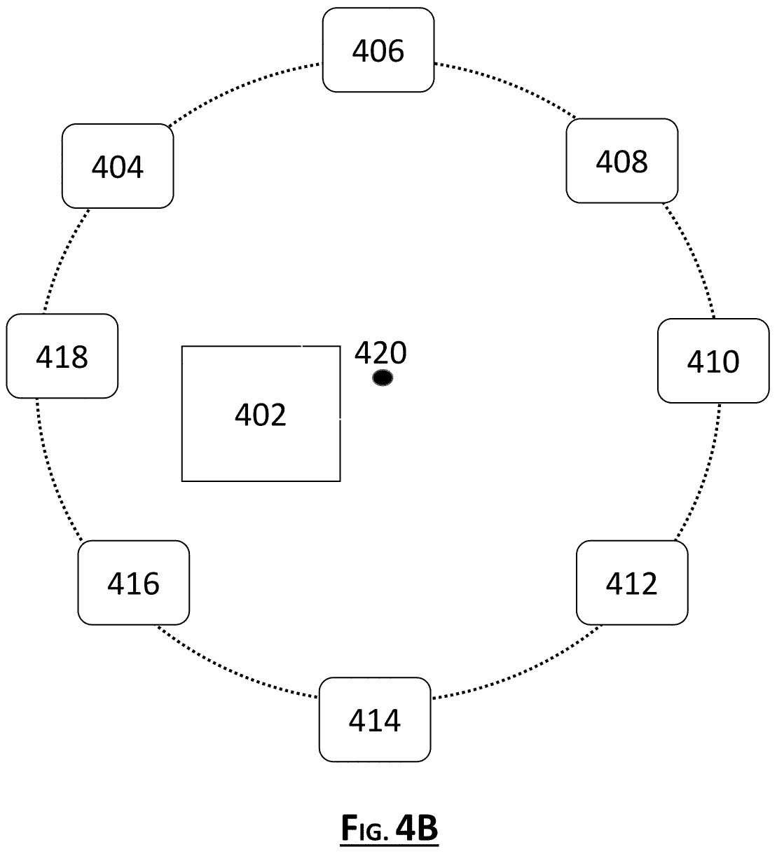

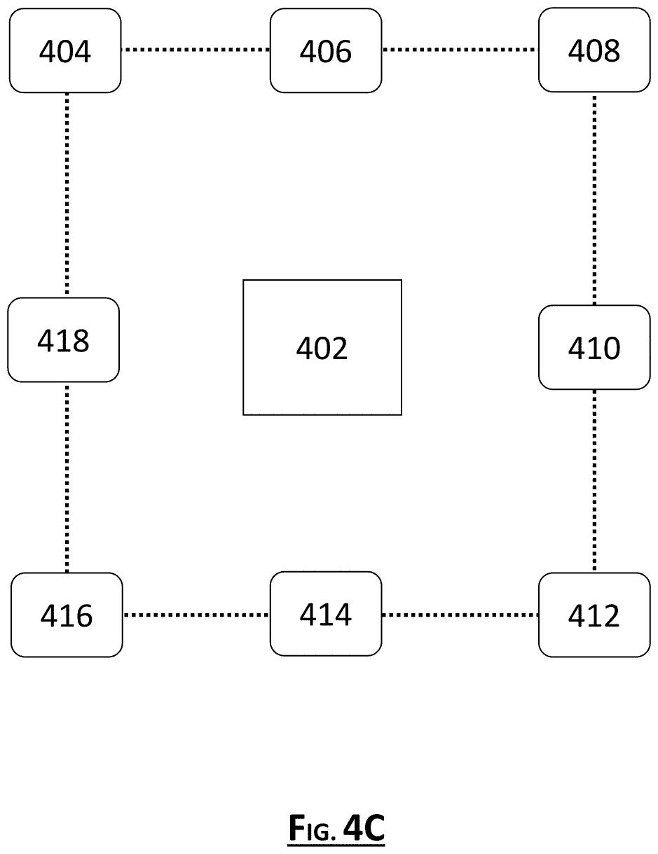

FIGS. 4A to 4C are schematic views of an imaging system with a plurality of RF applicator elements 404, 406, 408, 410, 412, 414, 416, 418. In FIG. 4A, each applicator element is at a constant radial distance from a thermoacoustic transducer 402 and are arranged in a symmetric pattern. In FIG. 4B, RF applicator elements 404, 406, 408, 410, 412, 414, 416, 418 are positioned in a circle, but the thermoacoustic transducer 402 is offset from a central location 420. In FIG. 4C, each RF applicator element is arranged in a rectangle around the thermoacoustic transducer 402. As shown in the examples, the RF applicator elements can be arranged in a symmetric pattern, but it is intended that asymmetric patterns of RF applicator elements may also be utilized.

The schematic views shown in FIGS. 4A to 4C are merely an example and can be extended to a smaller or larger number of applicator elements, such as 4, 16, or 32, each at a constant radial distance from the thermoacoustic transducer 402. The RF applicator elements 404, 406, 408, 410, 412, 414, 416, 418 are connected to an RF source (not shown). The system can adjust parameters of the RF applicator elements to steer or change the RF illumination field for generating a thermoacoustic signal in a more desired location.

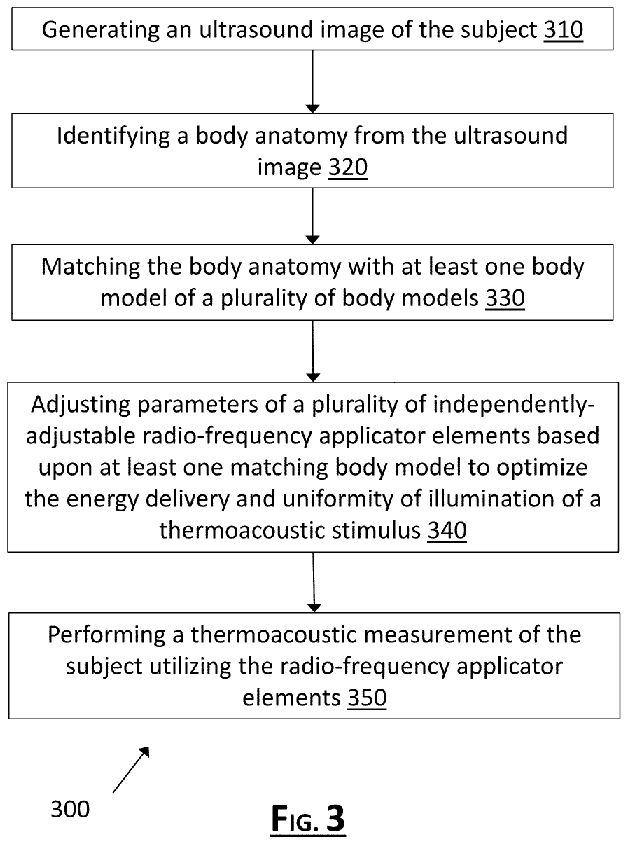

FIG. 3 is an embodiment of a method 300 for providing an image of a subject by utilizing an array of transmission elements (such as RF applicator elements) in a thermoacoustic imaging apparatus.

In step 310, a processor (e.g., a processor of the computing device) of the imaging system generates an ultrasound image of the subject, such as by utilizing the ultrasound transducer array of the ultrasound imaging system.

In step 320, a processor of the imaging system identifies a body anatomy from the ultrasound image. The processor can identify a body anatomy based on the generated image of the ultrasound imaging system. The body anatomy may be defined by attributes such as depth, width, and volume.

In step 330, a processor of the imaging system matches the body anatomy with at least one body model of a plurality of body models. The attributes are of the body anatomy are matched to at least one body model stored in a data store (e.g., the computing device) of the imaging system. The body models can include attributes for matching to the identified body anatomy, and the data records for each body model can correspond to desired parameters for each radio-frequency applicator element. These parameters can be based upon a simulation of different body anatomies to determine desired parameters for transmission by the thermoacoustic imaging system. The simulations can be virtual tests that can generate data records that include the attributes of the body anatomies along with the desired radio-frequency applicator element attributes. The data records can be stored in a lookup table that is queried by the processor to determine the appropriate parameters for that body anatomy.

In step 340, a processor of the imaging system adjusts parameters of a plurality of independently-adjustable radio-frequency applicator elements based upon at least one matching body model to optimize the energy delivery and uniformity of illumination of a thermoacoustic stimulus in a region of interest and to minimize thermoacoustic stimulus that would generate thermoacoustic artifacts that could interfere with the thermoacoustic response of interest. The adjustment can occur automatically once the body anatomy is matched to a body model. The radio-frequency applicator elements can be adjusted independently or in sets of one or more radio-frequency applicator elements. The radio-frequency applicator elements can be adjusted simultaneously or in a predetermined order. For example, upon determining desired parameters from a matching body model, the imaging system can adjust the amplitude and phase of each radio-frequency applicator element to steer the beam to the identified body anatomy. Although the examples herein discuss that multiple parameters can be adjusted, it is intended that the imaging system can adjust a single parameter for one or more radio-frequency applicator elements.

In an alternative embodiment, the processor of the imaging system can adjust at least one parameter of the independently-adjustable radio-frequency applicator elements without first utilizing an ultrasound image. The imaging system can iteratively adjust the parameters (e.g., changing the tuning) until an optimized signal is achieved.

In step 350, a processor of the imaging system performs a thermoacoustic measurement of the subject utilizing the radio-frequency applicator elements. The thermoacoustic measurement can calculate a value within the subject, such as a fat concentration. By steering the radio-frequency applicator elements based on the body anatomy, the imaging system can achieve a more ideal beam for a calculation without reflections, hot spots, or blocked areas.

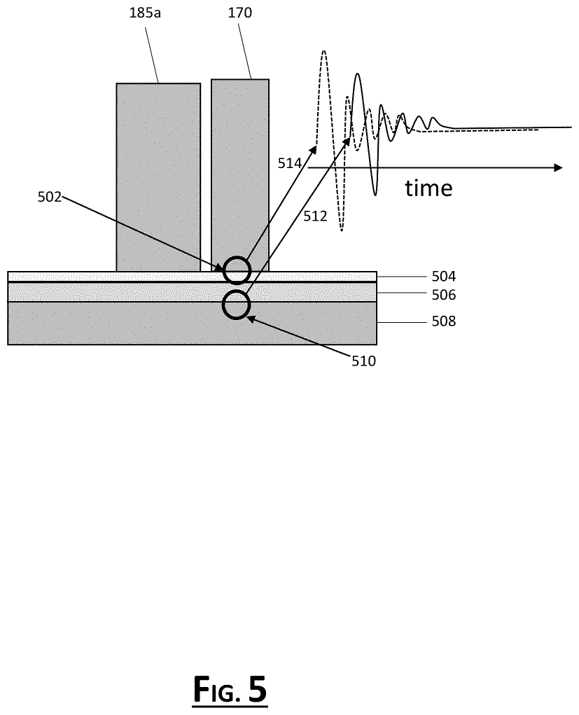

FIG. 5 shows thermoacoustic artifact generation at a transducer-skin boundary with a fat/muscle signal of interest. RF applicator(s) 185a pulses energy that is partially absorbed in the skin 504, fat 506, and muscle 508 layers. A signal of interest 512 is generated at boundary location 510 and is caused by different energy absorption rates between the fat layer 506 and muscle layer 508. An artifact signal 514 is generated at boundary location 502 and is caused by the skin 504 absorbing energy and vibrating against the transducer 170. The artifact signal 514 at boundary location 502 is still ringing down (decaying over time) when the signal of interest 512 is received at the transducer 170. Hence, artifact signal 514 distorts signal of interest 512, since the signal received at the transducer at a selected time is a summation of artifact signal 514 and signal of interest 512. Embodiments disclosed herein aim to adjust artifact signal 514 amplitude, phase, and decay time to mitigate the effect on signal of interest 512. Adjusting artifact signal 514 can be accomplished by adjusting RF applicator(s) 185a, e.g., adjustable in at least amplitude and phase, and more generally also adjustable in waveform, frequency, and polarization. Further, each applicator element can be adjustable to match a complex impedance of a body (e.g., patient) at its location, to maximize power transmission and minimize reflected power to optimize the image.

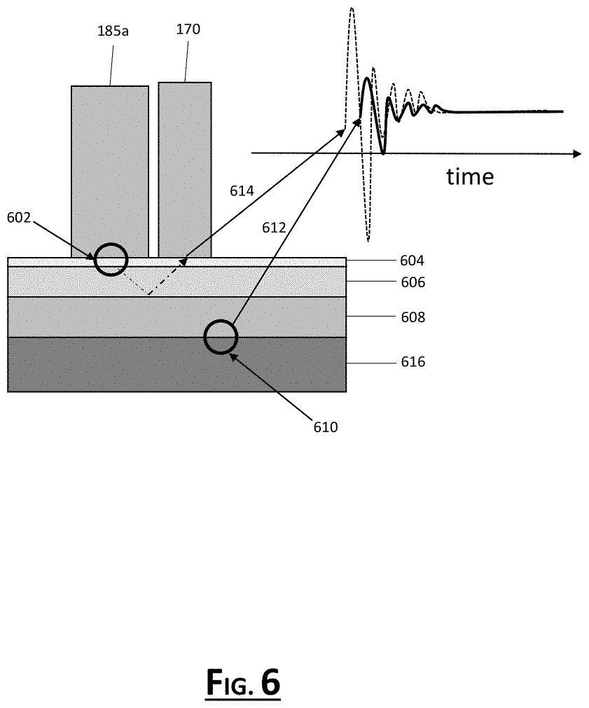

FIG. 6 shows thermoacoustic artifact generation at an applicator-skin boundary and a muscle/liver signal of interest. RF applicator(s) 185a pulses energy that is partially absorbed in the skin 604, fat 606, muscle 608, and liver 616 layers. The signal of interest 612 is generated at boundary location 610 and is caused by different energy absorption rates between the muscle layer 508 and liver layer 616. An artifact signal 614 is generated at boundary location 602 and is caused by the skin 604 absorbing energy and vibrating against RF applicator(s) 185a. In this example, artifact signal 614 reflects from the interface between the fat 606 and muscle 608 layer and is received at the transducer 170. The reflection delays artifact signal 614, causing artifact signal 614 to arrive at roughly the same time as signal of interest 610, depending upon a subject's anatomy. This will distort the received signal at the transducer for the signal of interest 610 similar to the example illustrated in FIG. 5.

A thermoacoustic body model is comprised of one or more artifacts (such as artifact signals 514 or 614). A user of the thermoacoustic system could expect artifact signal(s) to be generated based upon the body model (e.g. physical characteristics, temperature) of a person upon whom a thermoacoustic measurement is being conducted. The artifacts can then be minimized by utilizing the best RF applicator(s) 185a configuration for that particular body model. In addition, signals of interest 512 and 612 would be maximized by utilizing the RF applicator(s) 185a configuration for that particular body model.

Although embodiments have been described above with reference to the accompanying drawings, those of skill in the art will appreciate that variations and modifications may be made without departing from the scope thereof as defined by the appended claims.

What is claimed is:

1. A method for providing an image of a subject by utilizing an array of transmission elements in an imaging system, the method comprising:
generating an ultrasound image of the subject with an ultrasound apparatus;
identifying a body anatomy from the ultrasound image;
matching the body anatomy with at least one body model of a plurality of body models;
adjusting parameters of a plurality of independently-adjustable radio-frequency applicator elements based upon at the least one matched body model to optimize the energy delivery and uniformity of illumination of a thermoacoustic stimulus in a specific region of interest and minimize thermoacoustic stimuli outside of the specific region of interest; and
performing, by a processor, a thermoacoustic measurement of the subject utilizing the radio-frequency applicator elements.

2. The method of claim 1, wherein adjusting parameters of a plurality of independently-adjustable radio-frequency applicator elements comprises steering a beam from a first location to a second location based on the body anatomy.

3. The method of claim 1, wherein adjusting parameters comprises calculating a revised parameter based on a depth, width, and volume of the matching body model.

4. The method of claim 1, wherein adjusting parameters comprises identifying parameters in a lookup table based upon the matching body model.

5. The method of claim 1, wherein the parameters are automatically adjusted for transmission upon matching with at least one body model.

6. The method of claim 1, wherein adjusting parameters uses amplitude and phase values for each radio-frequency applicator element from a simulation of the matching body model.

7. The method of claim 1, wherein each radio-frequency applicator element is adjusted simultaneously.

8. The method of claim 1, wherein the radio-frequency applicator elements are arranged in a circular array.

9. The method of claim 1, wherein the radio-frequency applicator elements are arranged in a symmetrical array.

10. The method of claim 1, wherein the radio-frequency applicator elements are arranged with an offset from a center location.

11. The method of claim 1, wherein the parameters of each applicator element are determined by a respective shaped illumination field.

12. The method of claim 1, wherein the parameters are selected from the group consisting of an amplitude, a phase, a frequency, a polarization, a waveform, and an input impedance.

13. The method of claim 1, wherein adjusting parameters of the plurality of independently-adjustable radio-frequency applicator elements is based upon the at the least one matched body model and the ultrasound image of the subject to optimize the energy delivery and uniformity of illumination of the thermoacoustic stimulus in the specific region of interest and minimize thermoacoustic stimuli outside of the specific region of interest.

14. A method for providing an image of a subject by utilizing an array of transmission elements in a thermoacoustic imaging apparatus, the method comprising:
generating, by a processor, an ultrasound image of the subject;
adjusting, by the processor, parameters of a plurality of independently-adjustable radio-frequency applicator elements based upon the ultrasound image of the subject to optimize the energy delivery and uniformity of illumination of a thermoacoustic stimulus in a specific region of interest and minimize thermoacoustic stimuli outside of the specific region of interest; and
performing, by the processor, a thermoacoustic measurement of the subject utilizing the radio-frequency applicator elements.

15. A thermoacoustic imaging system comprising:
a set of applicator elements, each applicator element driven by an independent amplifier, wherein each independent amplifier has an adjustable phase and amplitude for each applicator element channel; and
the independent amplifier of each applicator element configured to adjust phase and amplitude for each applicator element of each channel to optimize a uniformity of energy deposition over a target volume, and configured to adjust each applicator element to maximize energy absorption in the target volume, thereby steering the set of applicator elements to the target volume.

16. The thermoacoustic imaging system of claim 15, further comprising an ultrasound transducer configured to generate an ultrasound image of the target volume, wherein the independent amplifier adjusts each applicator element based upon the ultrasound image of the target volume.

17. The thermoacoustic imaging system of claim 16, further comprising a processor configured to compare a body anatomy from the ultrasound image with at least one body model of a plurality of body models.

18. The thermoacoustic imaging system of claim 15, wherein the set of applicator elements is configured to perform a thermoacoustic measurement of a value of the target volume.

19. The thermoacoustic imaging system of claim 18, wherein the value is a fat concentration.

20. The thermoacoustic imaging system of claim 15, wherein the set of applicator elements are arranged in a circular array, symmetrical array, or with an offset from a center location.
